(19) United States
(12) Patent Application Publication
(10) Pub. No.: US 2020/0093658 A1
(43) Pub. Date: Mar. 26, 2020

(54) Diaper with Removable Wipe Pouch

(71) Applicant: Larry Copeland, San Diego, CA (US)

(72) Inventor: Larry Copeland, San Diego, CA (US)

(21) Appl. No.: 16/535,333

(22) Filed: Aug. 8, 2019

Related U.S. Application Data
(60) Provisional application No. 62/734,650, filed on Sep. 21, 2018.

(51) Int. Cl. A61F 13/84; A61F 13/49; A61F 13/62

(52) U.S. Cl. CPC A61F 13/84; A61F 13/49017; A61F 2013/49087; A61F 13/49058; A61F 2013/49093; A61F 13/622

(57) ABSTRACT

A diaper with removable wipe pouch. The diaper includes a flexible base having a front panel and a rear panel. The flexible base is movable between an open configuration and a closed configuration, wherein the front panel is secured to the rear panel when in the closed configuration, such that an upper opening is defined. The flexible base further includes arcuate lateral edges defining a pair of leg openings when the flexible base is in the closed configuration. A pouch is removably secured to the rear panel, wherein at least one cleaning wipe is disposed within an interior volume thereof. The pouch is secured to the rear panel via a continuous perforated seam disposed about a perimeter of the pouch.

### CROSS REFERENCE TO RELATED APPLICATIONS

This application claims the benefit of U.S. Provisional Application No. 62/734,650 filed on Sep. 21, 2018. The above identified patent application is herein incorporated by reference in its entirety to provide continuity of disclosure.

### BACKGROUND OF THE INVENTION

The present invention relates to a diaper with a removable wipe pouch. More particularly, the present invention pertains to a diaper having a removable wipe pouch secured thereto via a continuous perforated seam about a perimeter of the wipe pouch.

Many children, as well as some adults, suffer from incontinence, requiring the use of a diaper to prevent problems caused by a sudden loss of bowel control. Typically, when a user changes a dirty diaper, a cleaning wipe is used to remove any mess remaining on the individual before applying a clean diaper. These cleaning wipes are typically stored in a separate container, such as a diaper bag, purse, backpack, or the like, which must be carried in the event a diaper must be changed. Sometimes a user may forget to bring sufficient cleaning wipes, or otherwise run out, and have no convenient method of cleaning the wearer of the diaper. Therefore, there is a need for a diaper that includes a removable wipe pouch thereon, such that wipes are readily available at all times during a diaper change.

In light of the devices disclosed in the known art, it is submitted that the present invention substantially diverges in design elements from the known art and consequently it is clear that there is a need in the art for an improvement to existing diapers. In this regard, the instant invention substantially fulfills these needs.

### SUMMARY OF THE INVENTION

In view of the foregoing disadvantages inherent in the known types of diapers now present in the known art, the present invention provides a diaper with a removable wipe pouch wherein the same can be utilized for providing convenience for the user when cleaning a child when changing a diaper.

The present system comprises a flexible base having a front panel and a rear panel, wherein the flexible base is selectively movable between an open configuration and a closed configuration. When in the closed configuration, the front panel is removably secured to the rear panel, thereby defining an upper opening. The flexible base further comprises arcuate lateral edges defining a pair of leg openings when the flexible base is in the closed configuration. A pouch defining an interior volume is removably secured to an exterior of the rear panel, wherein at least one cleaning wipe is disposed within the interior volume. The pouch is removably secured to the rear panel via a continuous perforated seam disposed about a perimeter of the pouch. In some embodiments, a pair of straps extend from opposing sides of an upper end of the rear panel, wherein the pair of straps are removably secured to the front panel when the flexible base is in the closed configuration.

In some embodiments, the pouch further comprises a closure flap extending from an upper side thereof, wherein the closure flap forms a seal when secured to an exterior of the pouch. In another embodiment, the pouch comprises a fluid impermeable material. In other embodiments, an elastic material is disposed through each of the arcuate lateral edges, the elastic material biasing the leg openings radially inward. In yet another embodiment, a fastener is disposed on opposing sides of the rear panel, the fastener removably securable to a complementary fastener disposed on the front panel. In other embodiments, the fastener is disposed on an interior of each of the pair of straps. In some embodiments, the fastener comprises a hook and loop fastener. In another embodiment, an interior of the flexible base is lined with an absorbent and padded material. In other embodiments, the interior volume contains between three and five cleaning wipes. In yet another embodiment, the cleaning wipes are further impregnated with a cleaning fluid.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the diaper with removable wipe pouch in a closed configuration.

FIG. 2 shows a top plan view of an embodiment of the diaper with removable wipe pouch in an open configuration.

FIG. 3 shows an exploded view of an embodiment of the diaper with removable wipe pouch.

### DETAILED DESCRIPTION OF THE INVENTION

Reference is made herein to the attached drawings. Like reference numerals are used throughout the drawings to depict like or similar elements of the diaper with removable wipe pouch. The figures are intended for representative purposes only and should not be considered to be limiting in any respect.

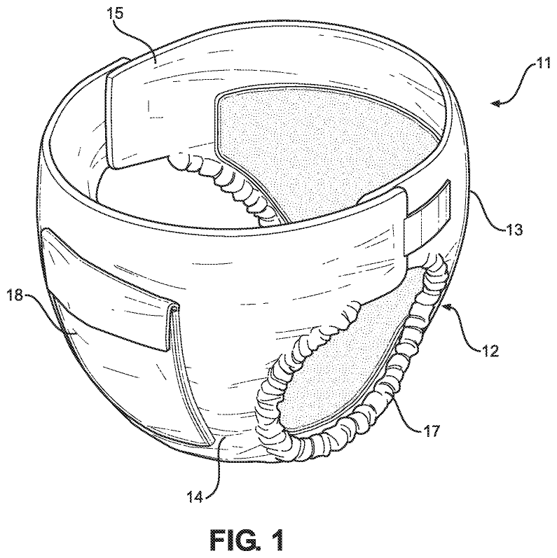

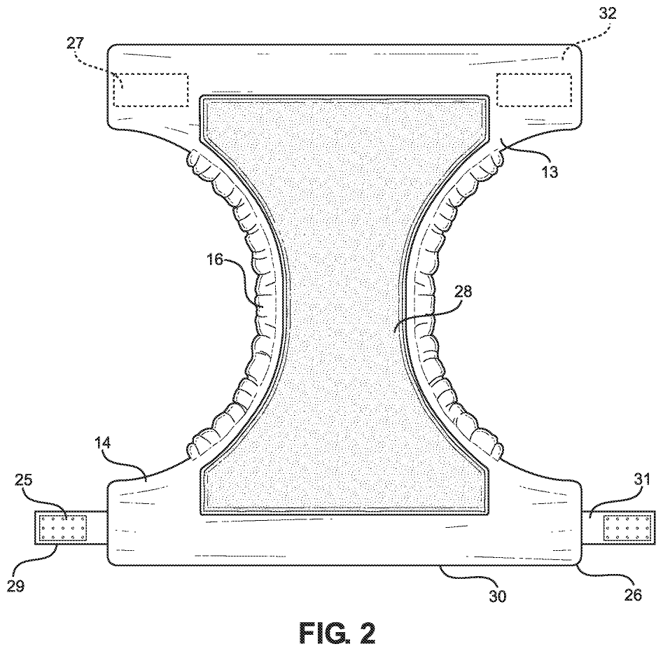

Referring now to FIG. 1, there is shown a perspective view of an embodiment of the diaper with removable wipe pouch in a closed configuration. The diaper with removable wipe pouch 11 comprises a flexible base 12 having a front panel 13 opposite a rear panel 14. The front panel 13 and the rear panel 14 are selectively movable between an open configuration (as shown in FIG. 2) and a closed configuration (as shown in FIG. 1). When in the closed configuration, a pair of leg openings 17 are defined opposite an upper opening 15, wherein each of the pair of leg openings 17 are configured to receive a leg of the wearer therethrough, and the upper opening 15 is configured to secure about a wearer's waist. A pouch 18 is disposed on an exterior surface of the rear panel 14, wherein the pouch 18 is configured to store one or more cleaning wipes therein. In this way, the user is provided a readily accessible source of cleaning wipes, such that the user can utilize the wipes to clean the wearer when changing a soiled diaper.

Referring now to FIG. 2, there is shown a top plan view of an embodiment of the diaper with removable wipe pouch in an open configuration. In the illustrated embodiment, the diaper with removable wipe pouch further comprises a pair of arcuate lateral edges 16 extending between the front panel 13 and the rear panel 14, wherein the pair of arcuate lateral edges 16 define the pair of leg openings when the flexible base is in the closed configuration. In some embodiments, the pair of arcuate lateral edges 16 further comprise an elastic material therein, such that the pair of leg openings are biased radially inwardly when the flexible base is in the closed position. In this way, the pair of leg openings constrict about the wearer's legs, minimizing the risk of leakage therethrough when the diaper is soiled.

In the illustrated embodiment, the flexible base further comprises an interior liner 28 disposed on an interior surface thereof, wherein the interior liner 28 comprises an absorbent and padded material. The interior liner 28 extends from the front panel 13 and onto the rear panel, such that an entirety of the interior surface is covered thereby. In this way, the interior liner 28 is properly positioned to capture and absorb any waste, such that the leakage or the like is prevented. As the interior liner 28 further comprises a padded material, comfort of the user is increased, minimizing any rashes, chafing, or the like from forming on the wearer due to friction. In the illustrated embodiment, a perimeter of the interior liner 28 conforms to a perimeter of the flexible base from the front panel 13, along the pair of arcuate lateral edges 16, and onto the rear panel 14. This arrangement ensures maximal coverage of the flexible base.

The rear panel 14 is removably securable to the front panel 13 via a fastener 25 disposed on the rear panel 14, the fastener 25 removably securable to a complementary fastener 27 disposed on an exterior surface 32 of the front panel 13. In the illustrated embodiment, the fastener 25 is disposed on an interior surface 31 of each of a pair of straps 29 extending from opposing sides 26 an upper end 30 of the rear panel 14. In some embodiments, the complementary fasteners 27 comprise a greater length than the fasteners 25, such that the fastener 25 is adjustably positioned along the complementary fastener 27 to adjust the size of the upper opening. In another embodiment, each of the pair of straps 29 comprises an elastic material to similarly allow the user to adjustably secure the diaper to the wearer.

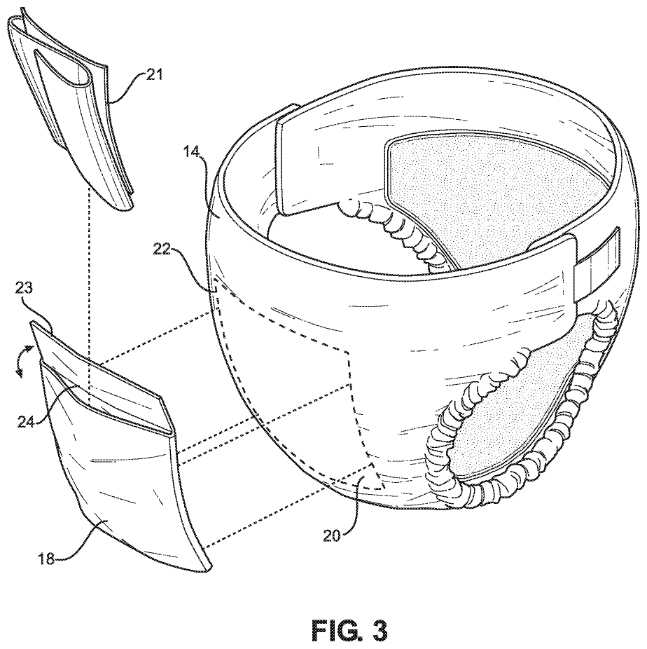

Referring now to FIG. 3, there is shown an exploded view of an embodiment of the diaper with removable wipe pouch. The pouch 18 is removably secured to the rear panel 14 of the flexible base. The pouch 18 defines an interior volume, wherein at least one cleaning wipe 21 is disposed within the interior volume. In some embodiments, between three to five cleaning wipes 21 are stored within the interior volume, such that a user is provided with sufficient wipes 21 to thoroughly clean the wearer when changing a soiled diaper. In some embodiments, the cleaning wipes 21 are impregnated with a cleaning fluid configured to aid a user in cleaning the wearer when changing a soiled diaper. Additionally, in some embodiments, the pouch 18 comprises a fluid impermeable material, such that the moisture from the cleaning wipes 21 does not contact the flexible base of the diaper, or alternatively, any waste seepage through the flexible base does not contaminate the cleaning wipes 21 within the pouch 18.

In the illustrated embodiment, the pouch 18 further comprises a closure flap 23 is pivotally affixed to the pouch 18 along an upper edge 24 thereof. The closure flap 23 is configured to removably secure to an exterior of the pouch 18 when in the closed position, such that a seal if formed thereby. The seal can be both watertight and airtight, such that the seal effectively prevents moisture within the pouch 18 from escaping or evaporating, thereby preserving the moisture of the cleaning wipes 21 within the interior volume of the pouch 18. In some embodiments, the closure flap 23 utilizes a resealable adhesive, such that one or more cleaning wipes 21 may be used at a first point, the closure flap 23 resealed against the exterior of the pouch 18, and the remaining cleaning wipes 21 used at a second point without the cleaning wipes 21 losing significant cleaning fluid due to evaporation or other drying effects.

The pouch 18 is removably secured to an exterior surface 20 of the rear panel 14 via a continuous perforated seam 22 extending along a perimeter of the pouch 18. The continuous perforated seam 22 allows the pouch 18 to be securely held in place while the diaper is being worn, while still allowing easy removal when the cleaning wipes 21 are needed. In this way, the user can remove the pouch 18 from the diaper, such that the user is not required to remain in close proximity to the soiled diaper once removed from the wearer. Furthermore, the removable pouch 18 allows the user to store any additional or remaining cleaning wipes 21 in an alternate container, such as a purse, pocket, or the like for future use.

In one exemplary use, once the diaper is soiled, the user can remove the pouch 18 from the rear panel 14 by tearing along the continuous perforated seam 22. Once removed from the rear panel, the user can remove the soiled diaper from the wearer and dispose of the diaper in a waste receptacle. In order to clean the wearer before the application of a fresh diaper, the pouch 18 can be opened by removing the closure flap 23 from the exterior surface of the pouch 18, providing access to the interior volume. One or more cleaning wipes 21 can then be removed from the pouch 18 to clean the soiled area of the wearer. Once cleaned, the fresh diaper can be applied, and any unused cleaning wipes 21 can be stored for future use by resealing the closure flap 23 against the exterior surface of the pouch 18.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A diaper with removable wipe pouch, comprising:
a flexible base having a front panel and a rear panel;
wherein the flexible base is selectively movable between an open configuration and a closed configuration, wherein the front panel is removably secured to the rear panel when in the closed configuration, thereby defining an upper opening;
wherein the flexible base further comprises arcuate lateral edges defining a pair of leg openings when the flexible base is in the closed configuration;
a pouch defining an interior volume removably secured to an exterior of the rear panel;
wherein at least one cleaning wipe is disposed within the interior volume;
wherein the pouch is removably secured to the rear panel via a continuous perforated seam disposed about a perimeter of the pouch.

2. The diaper with removable wipe pouch of claim 1, wherein the pouch further comprises a closure flap extending from an upper side thereof, wherein the closure flap forms a seal when secured to an exterior of the pouch.

3. The diaper with removable wipe pouch of claim 1, wherein the pouch comprises a fluid impermeable material.

4. The diaper with removable wipe pouch of claim 1, further comprising an elastic material through each of the arcuate lateral edges, the elastic material biasing the leg openings radially inward.

5. The diaper with removable wipe pouch of claim 1, further comprising a fastener disposed on opposing sides of the rear panel, the fastener removably securable to a complementary fastener disposed on the front panel.

6. The diaper with removable wipe pouch of claim 5, wherein the fastener comprises a hook and loop fastener.

7. The diaper with removable wipe pouch of claim 1, wherein an interior of the flexible base is lined with an absorbent and padded material.

8. The diaper with removable wipe pouch of claim 1, wherein the interior volume contains between three and five cleaning wipes.

9. The diaper with removable wipe pouch of claim 1, wherein the cleaning wipes are further impregnated with a cleaning fluid.

10. A diaper with removable wipe pouch, comprising:
a flexible base having a front panel and a rear panel;
a pair of straps extending from opposing sides of an upper end of the rear panel;
wherein the flexible base is selectively movable between an open configuration and a closed configuration, wherein the front panel is removably secured to the rear panel when in the closed configuration, thereby defining an upper opening;
wherein the flexible base further comprises arcuate lateral edges defining a pair of leg openings when the flexible base is in the closed configuration;
a pouch defining an interior volume removably secured to an exterior of the rear panel;
wherein at least one cleaning wipe is disposed within the interior volume;
wherein the pouch is removably secured to the rear panel via a continuous perforated seam disposed about a perimeter of the pouch.

11. The diaper with removable wipe pouch of claim 10, wherein the pouch further comprises a closure flap extending from an upper side thereof, wherein the closure flap forms a seal when secured to an exterior of the pouch.

12. The diaper with removable wipe pouch of claim 10, wherein the pouch comprises a fluid impermeable material.

13. The diaper with removable wipe pouch of claim 10, further comprising an elastic material through each of the arcuate lateral edges, the elastic material biasing the leg openings radially inward.

14. The diaper with removable wipe pouch of claim 10, further comprising a fastener disposed on an interior surface of each of the pair of straps, the fastener removably securable to a complementary fastener disposed on an exterior surface of the front panel.

15. The diaper with removable wipe pouch of claim 14, wherein the fastener comprises a hook and loop fastener.

16. The diaper with removable wipe pouch of claim 10, wherein an interior of the flexible base is lined with an absorbent and padded material.

17. The diaper with removable wipe pouch of claim 10, wherein the interior volume contains between three and five cleaning wipes.

18. The diaper with removable wipe pouch of claim 10, wherein the cleaning wipes are further impregnated with a cleaning fluid.
